(19) United States
(12) Patent Application Publication
(10) Pub. No.: US 2018/0214027 A1
(43) Pub. Date: Aug. 2, 2018

(54) ACOUSTIC WAVE MEASURING APPARATUS AND CONTROL METHOD THEREOF

(71) Applicant: CANON KABUSHIKI KAISHA, Tokyo (JP)

(72) Inventor: Yoshihiro Hirata, Fuchu-shi (JP)

(21) Appl. No.: 15/865,823

(22) Filed: Jan. 9, 2018

(30) Foreign Application Priority Data
Jan. 27, 2017 (JP) 2017-012671

(51) Int. Cl. A61B 5/00; H03H 9/02; G01S 15/89; G10K 11/36

(52) U.S. Cl. CPC A61B 5/0095; G10K 11/36; G01S 15/8965; H03H 9/02968

(57) ABSTRACT

An acoustic wave measuring apparatus generates a characteristics information distribution of the inside of an object by using an acoustic wave propagating from the object, and includes a receiving unit that receives the acoustic wave, a first imaging unit that images a first area of the object from a first direction, and acquires an image of the first area, a second imaging unit that images a second area smaller than the first area of the object from a second direction different from the first direction, and acquires a local image, a designating unit that receives, from a user, designation of a position where the imaging is performed by the second imaging unit in the image of the first area captured by the first imaging unit.

### BACKGROUND OF THE INVENTION

### Field of the Invention

The present invention relates to an acoustic wave measuring apparatus and a control method thereof.

### Description of the Related Art

There is a technique called photoacoustic imaging (PAI) that images a tissue inside an object by using a photoacoustic wave generated from a light absorber inside the object by irradiating the object with light generated from a light source.

Japanese Patent Application Laid-open No. 2016-137053 describes an object information acquiring apparatus that acquires the photoacoustic wave while scanning the object by moving a hemispherical probe relative to the object. Japanese Patent Application Laid-open No. 2016-137053 describes that it is possible to perform area designation of a region of interest while referring to an image captured by a camera, but does not clearly describe the position of the camera relative to the probe.
Patent Literature 1: Japanese Patent Application Laid-open No. 2016-137053

### SUMMARY OF THE INVENTION

In the case where the camera is provided in a structure in which the hemispherical probe is also provided, it is possible to perform imaging by the camera while performing measurement of the photoacoustic wave. However, in the case where a distance between the probe and the object is short, only part of the region of the object will probably come into a camera view. In this case, a user needs to search for a position where the user desires to perform measurement by moving the limited range of the camera view, and hence it is feared that the designation of the measurement position cannot be performed easily.

Note that the above problem is not limited to the photoacoustic imaging apparatus, and applies also to an ultrasonic echo apparatus.

The present invention has been made in view of the above problem. An object of the present invention is to allow easy designation of the measurement position or a measurement area.

The present invention provides an acoustic wave measuring apparatus for generating a characteristics information distribution of an inside of an object by using an acoustic wave propagating from the object, the acoustic wave measuring apparatus comprising:

a receiving unit configured to receive the acoustic wave;

a first imaging unit configured to image a first area of the object from a first direction, and acquire an image of the first area;

a second imaging unit configured to image an area smaller than the first area of the object from a second direction different from the first direction, and acquire a local image;

a designating unit configured to receive, from a user, designation of a position where the imaging is performed by the second imaging unit in the image of the first area captured by the first imaging unit, and receive, from the user, designation of a second area, the characteristics information distribution of which is generated in the local image captured by the second imaging unit; and

a generating unit configured to generate the characteristics information distribution by using the acoustic wave received by the receiving unit.

The present invention also provides a control method for an acoustic wave measuring apparatus for generating a characteristics information distribution of an inside of an object by using an acoustic wave propagating from the object, the acoustic wave measuring apparatus including:

a receiving unit that receives the acoustic wave;

a first imaging unit that images a first area of the object from a first direction, and acquires an image of the first area; and

a second imaging unit that images an area smaller than the first area of the object from a second direction different from the first direction, and acquires a local image,

the control method comprising:

receiving, from a user, designation of a position where the imaging is performed by the second imaging unit in the image of the first area captured by the first imaging unit, and receiving, from the user, designation of a second area, the characteristics information distribution of which is generated in the local image captured by the second imaging unit; and

generating the characteristics information distribution by using the acoustic wave received by the receiving unit.

According to the present invention, it is possible to easily designate the measurement position or the measurement area.

Further features of the present invention will become apparent from the following description of exemplary embodiments with reference to the attached drawings.

### BRIEF DESCRIPTION OF THE DRAWINGS

FIG. 1 is a cross-sectional view showing an example of a configuration of an acoustic wave measuring apparatus;

FIG. 2 is a view showing an example of a flow of acoustic wave measurement;

FIGS. 3A and 3B are views showing an example of an image captured by an wide-view imaging camera; and

FIGS. 4A to 4C are views showing examples of an image captured by a local imaging camera and designation of a measurement position.

### DESCRIPTION OF THE EMBODIMENTS

Hereinbelow, preferred embodiments of the present invention will be described with reference to the drawings. Note that the dimensions, materials, shapes, and relative arrangements of components described below should be appropriately changed according to the configuration of an apparatus to which the invention is applied and various conditions. Therefore, the scope of the invention is not limited to the description below.

The present invention relates to a technique for detecting an acoustic wave propagating from an object, and generating and acquiring characteristics information of the inside of the object. Therefore, the present invention is viewed as an acoustic wave measuring apparatus or a control method thereof, an object information acquiring method, or a signal processing method. In addition, the present invention may also be viewed as a program that causes an information processing apparatus including hardware resources such as a CPU and a memory to execute these methods. Further, the present invention is also viewed as a non-transitory computer-readable storage medium in which the program is stored.

The acoustic wave measuring apparatus of the present invention includes an apparatus that uses a photoacoustic effect to receive the acoustic wave generated inside the object by irradiating the object with light (electromagnetic wave), and acquire the characteristics information of the object as image data. In this case, the characteristics information is information on characteristic values that are generated by using a reception signal obtained by receiving a photoacoustic wave and correspond to a plurality of positions inside the object.

The characteristics information (photoacoustic characteristics information) deriving from an electrical signal (photoacoustic signal) acquired by photoacoustic measurement is a value in which the absorptance of light energy is reflected. The characteristics information includes, e.g., the generation source of the acoustic wave generated by light irradiation, an initial sound pressure in the object, or a light energy absorption density or an absorption coefficient derived from the initial sound pressure, or the concentration of a substance that constitutes a tissue. In addition, by determining oxygenated hemoglobin concentration and deoxygenated hemoglobin concentration as the substance concentration, it is possible to calculate an oxygen saturation distribution. Further, glucose concentration, collagen concentration, melanin concentration, and the volume fraction of fat or water are also determined.

The acoustic wave measuring apparatus of the present invention includes an apparatus that uses an ultrasonic echo technique to transmit an ultrasonic wave to the object, receive a reflected wave (echo wave) reflected inside the object, and acquire object information as the image data. The characteristics information (ultrasonic characteristics information) deriving from an electrical signal (ultrasonic echo signal) acquired by the ultrasonic echo apparatus is information in which a difference in the acoustic impedance of the tissue inside the object is reflected.

A two-dimensional or three-dimensional characteristics information distribution is obtained based on the characteristics information at each position inside the object. Distribution data can be generated as image data. The characteristics information may be determined not as numerical data but as distribution information at each position inside the object. That is, examples of the distribution information include an initial sound pressure distribution, an energy absorption density distribution, an absorption coefficient distribution, and an oxygen saturation distribution. In addition, an acoustic impedance distribution and distribution information indicative of the bloodstream can also be generated. Thus, the information based on the acoustic wave is visualized, and hence the present invention is also viewed as an acoustic wave imaging apparatus, a control method thereof, or a program.

The acoustic wave mentioned in the present invention is typically the ultrasonic wave, and includes an elastic wave called a sound wave or the acoustic wave. An electrical signal converted from the acoustic wave by a probe or the like is also referred to as an acoustic signal. Note that the description of the ultrasonic wave or the acoustic wave in the present specification is not intended to limit the wavelength of the elastic wave. The acoustic wave generated by the photoacoustic effect is referred to as the photoacoustic wave or a photo-ultrasonic wave. An electrical signal deriving from the photoacoustic wave is also referred to as a photoacoustic signal. An electrical signal deriving from the echo wave that is a transmitted ultrasonic wave reflected in the object is also referred to as the ultrasonic echo signal.

In the case where the apparatus of the present invention receives the photoacoustic wave, an ultrasonic echo, or both of them, the apparatus always measures the acoustic wave. Therefore, the apparatus of the present invention can be referred to as the acoustic wave measuring apparatus. The present invention is also viewed as the control method for the acoustic wave measuring apparatus.

### Embodiment 1

### Apparatus Overall Configuration

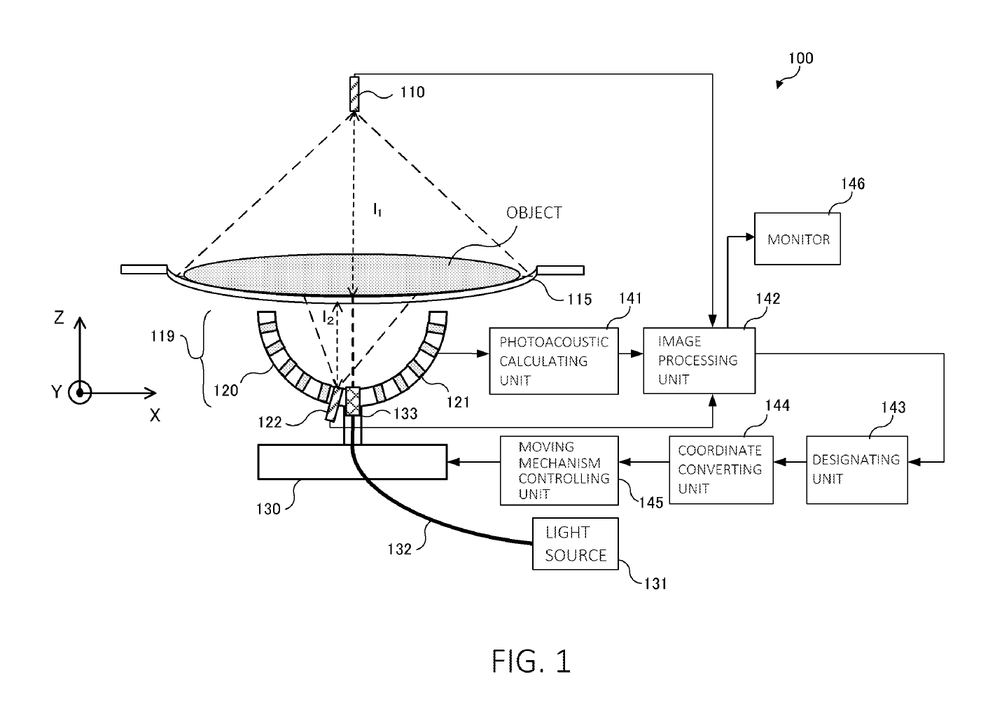

FIG. 1 is a view showing an example of a configuration of an acoustic wave measuring apparatus 100. FIG. 1 includes a cross-sectional view of a configuration related to measurement, and a block diagram of a configuration related to control. In the present embodiment, the acoustic wave measuring apparatus 100 is an apparatus that measures an acoustic wave emitted from the inside of a hand. The acoustic wave measuring apparatus 100 generates a photoacoustic image based on the measured acoustic wave. Note that, in the present embodiment, a description will be made on the assumption that an object is, e.g., the hand.

The acoustic wave measuring apparatus 100 has an wide-view imaging camera 110, a holding member 115, a probe 119, a local imaging camera 122, a moving mechanism 130, a light source 131, and a light transmission path 132. In addition, the acoustic wave measuring apparatus 100 has a photoacoustic calculating unit 141, an image processing unit 142, a designating unit 143, a coordinate converting unit 144, a moving mechanism controlling unit 145, and a monitor 146.

In addition, the probe 119 has a supporter 120, converting elements 121, and an emission opening 133. The probe 119 is capable of scanning a two-dimensional plane. It is assumed that the two-dimensional plane scanned by the probe 119 is an XY plane, and an axis perpendicular to the XY plane is a Z-axis. Hereinbelow, in the cross-sectional view in FIG. 1, an upward direction is referred to as a Z-axis positive direction, and a downward direction is referred to as a Z-axis negative direction. In the present embodiment, it is assumed that the acoustic wave is measured with the hand placed substantially parallel to the XY plane.

Hereinbelow, each configuration of the acoustic wave measuring apparatus 100 will be specifically described.

The holding member 115 has a curved surface, and is capable of placing the hand such that the entire hand is accommodated in the portion of the curved surface.

The holding member 115 holds the object in a state in which the object is placed on the holding member 115. Space between the object and the holding member 115 may be filled with an acoustic medium (not shown) such that the acoustic wave easily passes through the space. Examples of the acoustic medium may include water, oil, and gel.

As shown in FIG. 1, the wide-view imaging camera 110 is installed at a position spaced from the central portion of the holding member 115 by a distance l₁ in the Z-axis positive direction, and is capable of imaging the entire back of the hand. The wide-view imaging camera 110 outputs an image captured by the wide-view imaging camera 110 to the image processing unit 142 as image data. The wide-view imaging camera 110 corresponds to a first imaging unit of the present invention. The wide-view imaging camera 110 does not necessarily be capturing entire back of the hand, but has a wider field of view compared to the local imaging camera 122.

On the other hand, the supporter 120 is installed at a position in the Z-axis negative direction relative to the holding member 115. The supporter 120 is cup-shaped, and supports a plurality of the converting elements 121, the local imaging camera 122, and the emission opening 133 of light that is transmitted from the light source 131 via the light transmission path 132. The emission opening 133 corresponds to a light irradiating unit of the present invention. The wide-view imaging camera 110 is capable of imaging the area of the object larger than that of the local imaging camera 122, which will be described in detail later. The local imaging camera 122 images part of the object from a direction different from that of the wide-view imaging camera 110, and outputs an image captured by the local imaging camera 122 to the image processing unit 142 as image data. The local imaging camera 122 corresponds to a second imaging unit of the present invention. In an example in FIG. 1, the local imaging camera 122 is installed on a side opposite to the wide-view imaging camera 110 when viewed from the object.

The supporter 120 is cup-shaped, the bottom portion of the supporter 120 is fixed to the moving mechanism 130, and the opening side of the cup of the supporter 120 is directed to the holding member 115. For example, the moving mechanism 130 is a biaxial driving apparatus capable of tow-dimensional scanning. That is, the supporter 120 scans the above-described XY plane with the moving mechanism 130. Hereinbelow, it is assumed that the directions of the two axes are an X-axis and a Y-axis. In addition, a right direction in FIG. 1 is referred to as an X-axis positive direction, and a left direction therein is referred to as an X-axis negative direction. Further, a backward direction in FIG. 1 is referred to as a Y-axis positive direction, and a forward direction therein is referred to as a Y-axis negative direction. Note that the cross-sectional view in FIG. 1 corresponds to the X-axis direction and the Z-axis direction.

The moving mechanism 130 is constituted by combining, e.g., a linear guide (not shown), a feed screw mechanism (not shown), and a motor (not shown). Herein, the converting element 121 corresponds to a receiving unit of the present invention, and the supporter 120 corresponds to a supporting unit of the present invention.

The moving mechanism controlling unit 145 controls the moving mechanism 130 to move the supporter 120 on the XY plane and change the position relative to the object. In the present embodiment, with this, the local imaging camera 122 becomes capable of imaging each portion of the object. Note that the moving mechanism 130 is an example of a position moving unit.

The designating unit 143 receives the position where a local image is acquired in an wide-view image from a user, and also receives a measurement position designated by the user. Specifically, the designating unit 143 receives, from the user, designation of a second area imaged by the local imaging camera 122 in the wide-view image captured by the wide-view imaging camera 110. In addition, the designating unit 143 receives designation of the measurement position from the user by using the local image captured by the local imaging camera 122. Note that a specific example of the designation of the measurement position will be described later. Herein, the image captured by the wide-view imaging camera 110 is referred to as the wide-view image of the entire object, and the partial image of the object captured by the local imaging camera 122 is referred to as the local image.

The coordinate converting unit 144 converts the designated measurement position in the local image to a coordinate system corresponding to the XY plane described above. Hereinbelow, this coordinate system is referred to as an actual coordinate system. The moving mechanism controlling unit 145 moves the supporter 120 according to actual coordinates (X, Y) corresponding to the designated measurement position.

Note that space between the holding member 115 and the converting element 121 may be filled with an acoustic medium (not shown) that allows propagation of the acoustic wave. Examples of the acoustic medium may include water, oil, and gel.

The light transmission path 132 transmits pulsed light emitted from the light source 131 to the emission opening 133 at the end portion of the light transmission path 132 attached to the supporter 120, and irradiates the hand with the pulsed light from the emission opening 133. Note that the use of the light transmission path 132 is not limited to the case where the hand is irradiated with the pulsed light in the Z-axis positive direction, and the hand may be irradiated with the pulsed light after the light transmission path 132 is angled.

The light source 131 preferably generates the pulsed light having a pulse width of about 1 to 100 nanoseconds in order to generate the photoacoustic wave from the object efficiently. In addition, as its wavelength, the wavelength of about 600 nm to 1100 nm is preferable. As specific examples of the light source, an Nd:YAG laser, an alexandrite laser, a TiSa laser, and an OPO laser may be used. In addition, it is also possible to use a solid laser, a gas laser, a fiber laser, a dye laser, and a semiconductor laser. Further, an LED may be used as the light source. Note that the light source 131 may be provided integrally with the acoustic wave measuring apparatus 100 of the present embodiment, or may also be separated and provided as a separate component.

The timing of irradiation, the waveform, and the intensity are controlled by a light source controlling unit that is not shown. In addition, by using a wavelength variable laser capable of switching among a plurality of wavelengths, substance concentration information such as oxygen saturation and glucose concentration may be acquired.

When the object is irradiated with the pulsed light, the acoustic wave is generated from the inside of the object. The acoustic wave generated inside the object and emitted in the direction of the probe 119 reaches the converting elements 121 of the probe 119 via the holding member 115 and the acoustic medium. The individual converting elements 121 are disposed uniformly inside the cup-shaped supporter 120 such that the acoustic wave having reached the cup-shaped supporter 120 can be received in a large area. Each converting element 121 generates an electrical signal (hereinafter referred to as a PA signal) based on the received acoustic wave. Each converting element 121 transmits the generated PA signal to the photoacoustic calculating unit 141. The photoacoustic calculating unit 141 performs generation (image reconstruction) of photoacoustic image data by using the input PA signal. Note that the photoacoustic calculating unit 141 corresponds to a generating unit of the present invention. The image processing unit 142 generates display image data based on the photoacoustic image data, and displays the display image data on a monitor 146.

The imaging area of each camera will be described. In the present embodiment, the view angle of the wide-view imaging camera 110 is substantially similar to that of the local imaging camera 122, and hence the imaging area is larger as a distance to the object is longer.

The wide-view imaging camera 110 is installed at the position spaced from the central portion of the curved surface of the holding member 115 by the distance l₁ in the Z-axis positive direction so as to be able to image the entire back of the hand. Note that the use of the wide-view imaging camera 110 is not limited to the case where the entire back of the hand is imaged, and the wide-view imaging camera 110 may image an area that is larger than the local image to some extent, e.g., the area of about 80% of the back of the hand. Thus, the use of the wide-view imaging camera 110 is not limited to the case where the entire object is imaged, and the imaging area may appropriately be a relatively large area.

The local imaging camera 122 is installed at the position in the Z-axis negative direction when viewed from the holding member 115. The local imaging camera 122 is fixed to the supporter 120 that is positioned in the Z-axis negative direction when viewed from the holding member 115, and images the object locally. When the local imaging camera 122 is positioned immediately under the central portion of the curved surface of the holding member 115, a distance between the holding member 115 and the local imaging camera 122 is l₂. The distance l₁ between the wide-view imaging camera 110 and the holding member 115 is longer than the distance l₂ between the local imaging camera 122 and the holding member 115. The distance l₁ and the distance l₂ are designed according to an area ratio between the wide-view image and the local image. Note that, in the case where the view angle of the wide-view imaging camera 110 is wider than the view angle of the local imaging camera 122, the distance l₁ may be substantially equal to the distance l₂ or shorter than the distance l₂.

The local imaging camera 122 may perform the imaging by using, e.g., the position to which the pulsed light is applied by the light source 131 as the center of the imaging area. The local imaging camera 122 is fixed by the supporter 120, and hence the imaging area is changed in response to the movement of the supporter 120. As will be described later, the imaging area of the local imaging camera 122 is designated by the user.

Hereinbelow, the material and characteristics of each member will be described.

### Holding Member

Examples of preferable characteristics of the holding member 115 include thickness that allows the acoustic wave to easily pass through the holding member 115, transparency that allows light to pass through the holding member 115, and strength that allows the holding member 115 to bear the weight of the object. Examples of the material having these characteristics include polyethylene terephthalate (PET) and polyvinyl chloride (PVC).

Converting Element

A plurality of the converting elements 121 that receive the photoacoustic wave are disposed all over the inside of the cup-shaped supporter 120, and the probe 119 is thereby constituted.

The converting element 121 detects the acoustic wave, converts the acoustic wave to a signal such as an electrical signal, and outputs the signal. Any element such as an element that uses a piezoelectric phenomenon, an element that uses resonance of light, or an element that uses change of capacity may be used as the converting element 121 as long as the element is capable of detecting the acoustic wave. In the present embodiment, a plurality of the converting elements 121 are disposed, and are disposed such that the directions of the highest reception sensitivities of the individual converting elements 121 are different from each other. It is possible to receive the acoustic waves simultaneously at different locations by using the multi-dimensionally arrayed elements, and hence it is possible to improve image quality and reduce measurement time. As a more preferable mode, it is preferable to dispose a plurality of the converting elements 121 such that receiving surfaces of the converting elements 121 are directed to the center of the cup-shaped supporter 120.

Light Transmission Path

The pulsed light emitted from the light source 131 in FIG. 1 is guided to the object while being formed into a desired light distribution shape by the light transmission path 132. The light transmission path 132 may be an optical waveguide such as an optical fiber, an optical fiber bundle obtained by bundling the optical fibers, or an articulating arm in which a mirror or the like is incorporated into a lens-barrel. In addition, as the light transmission path 132, an optical component such as, typically, a lens, a mirror, or a diffuser may also be used.

Signal Processing by Photoacoustic Calculating Unit

The photoacoustic calculating unit 141 may include an AD conversion circuit (not shown) that digitizes an analog electrical signal, and an amplifier (not shown) that amplifies an electrical signal. A digital electrical signal obtained chronologically for each receiving element is output to and stored in a memory (not shown).

Photoacoustic Calculating Unit⋅Designating Unit⋅Coordinate Converting Unit⋅Moving Mechanism Controlling Unit⋅Image Processing Unit

Each of the functional sections of the photoacoustic calculating unit 141, the image processing unit 142, the designating unit 143, the coordinate converting unit 144, and the moving mechanism controlling unit 145 is implemented by executing a required program by a CPU (processor). The photoacoustic calculating unit 141, the image processing unit 142, the designating unit 143, the coordinate converting unit 144, and the moving mechanism controlling unit 145 may be configured as program modules that implement their respective functions in the same information processing apparatus. Alternatively, they may operate in different information processing apparatuses, and cooperate to execute information processing related to a photoacoustic imaging apparatus.

In addition, an inputting apparatus (e.g. a mouse, a keyboard, or a touch panel) of the information processing apparatus may be used as an inputting unit of the acoustic wave measuring apparatus 100. Examples of an input item from the inputting unit include the measurement position and the measurement area, a measurement parameter, and desired image quality.

When the photoacoustic calculating unit 141 performs image reconstruction, it is possible to use any method such as phasing addition or back projection.

Monitor

The monitor 146 displays the generated photoacoustic image, the image captured by the wide-view imaging camera 110, and the image captured by the local imaging camera 122. As the monitor 146, it is possible to use any displaying apparatus such as a liquid crystal display or an organic EL display. The monitor 146 may be integral with or separate from the acoustic wave measuring apparatus 100.

Measurement Flow

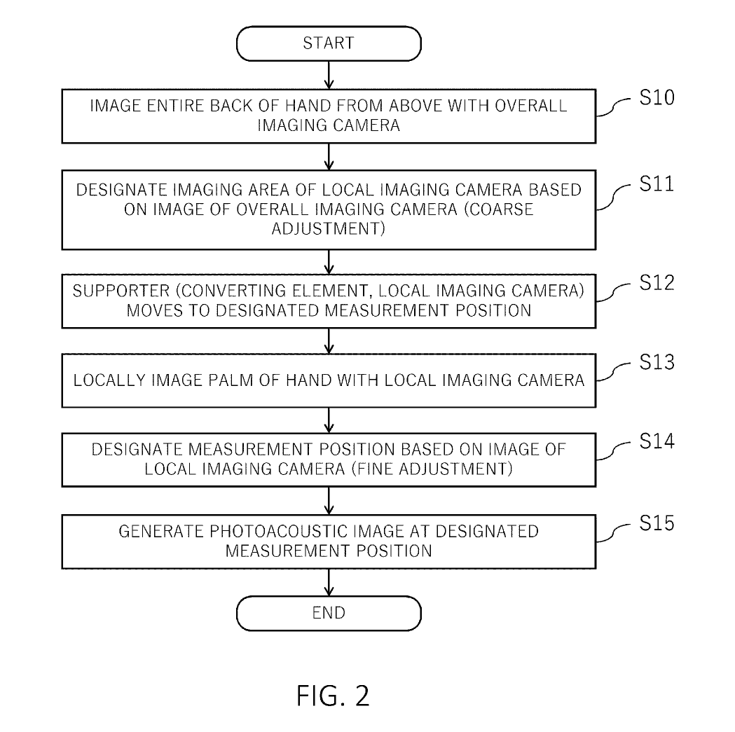

FIG. 2 is a view showing an example of a flow of acoustic wave measurement. Hereinbelow, the flow of processing of the acoustic wave measurement will be described. In Step S10, the wide-view imaging camera 110 images the entire back of the hand. The image processing unit 142 causes the monitor 146 to display the image captured by the wide-view imaging camera 110.

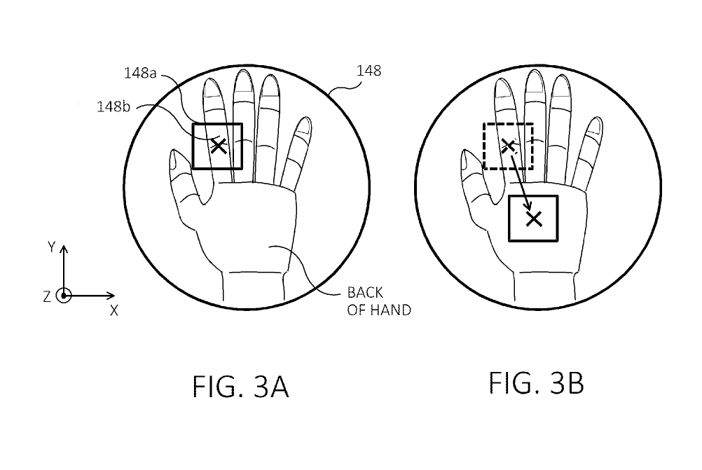

Each of FIGS. 3A and 3B shows an example of the image captured by the wide-view imaging camera 110. As shown in FIG. 3A, the entire back of the hand is included in an image 148 by the wide-view imaging camera 110. Note that the image 148 corresponds to the XY plane, a lateral direction in FIG. 3 corresponds to the X-axis, and a vertical direction therein corresponds to the Y-axis.

In Step S11, the designating unit 143 receives, from the user, the designation of the position of the area imaged by the local imaging camera 122 in the image 148 by the wide-view imaging camera 110 in FIG. 3A. Hereinbelow, rough adjustment of the position of the imaging area of the local imaging camera 122 is referred to as coarse adjustment.

In the image by the wide-view imaging camera 110 in FIG. 3A, an imaging area 148a of the local imaging camera 122 and a cursor 148b are displayed. The imaging area 148a shown in FIG. 3A indicates the imaging area of the local imaging camera 122. The cursor 148b indicates the center of the imaging area of the local imaging camera 122.

The designating unit 143 receives change of the position of the imaging area of the local imaging camera 122 from the user. The user can change the position of the imaging area of the local imaging camera 122 by moving the cursor 148b. For example, as shown in FIG. 3B, when the position of the cursor 148b is changed from the position near the base of the index finger to the position of the back of the hand, the designating unit 143 designates a region having the changed position of the cursor 148b at the center as the imaging area 148a of the local imaging camera 122. Note that, in the present embodiment, the designation of the imaging area 148a of the local imaging camera 122 is performed by using the cursor 148b, but the present invention is not limited thereto. For example, the designating unit 143 may receive the designation of the imaging area of the local imaging camera 122 by causing the user to numerically input the movement amount of the imaging area of the local imaging camera 122.

The coordinate converting unit 144 converts the position designated on the image of the wide-view imaging camera 110 to actual coordinates (X, Y). At this point, the coordinate converting unit 144 may perform the conversion of the coordinate by using a conversion formula or a correction table that is recorded in the memory in advance. Note that the conversion formula of the coordinate and the correction table thereof may be generated based on a parameter when the apparatus is designed or assembled.

In Step S12, the moving mechanism controlling unit 145 drives and controls the moving mechanism 130 such that the supporter 120 (the converting element 121, the local imaging camera 122) is moved to the designated actual coordinate position (X, Y).

In Step S13, the local imaging camera 122 fixed to the supporter 120 together with the converting element 121 locally images the palm of the hand at the designated actual coordinate position (X, Y) from the same side as the converting element 121. The image processing unit 142 receives the image by the local imaging camera 122, and causes the monitor 146 to display the image.

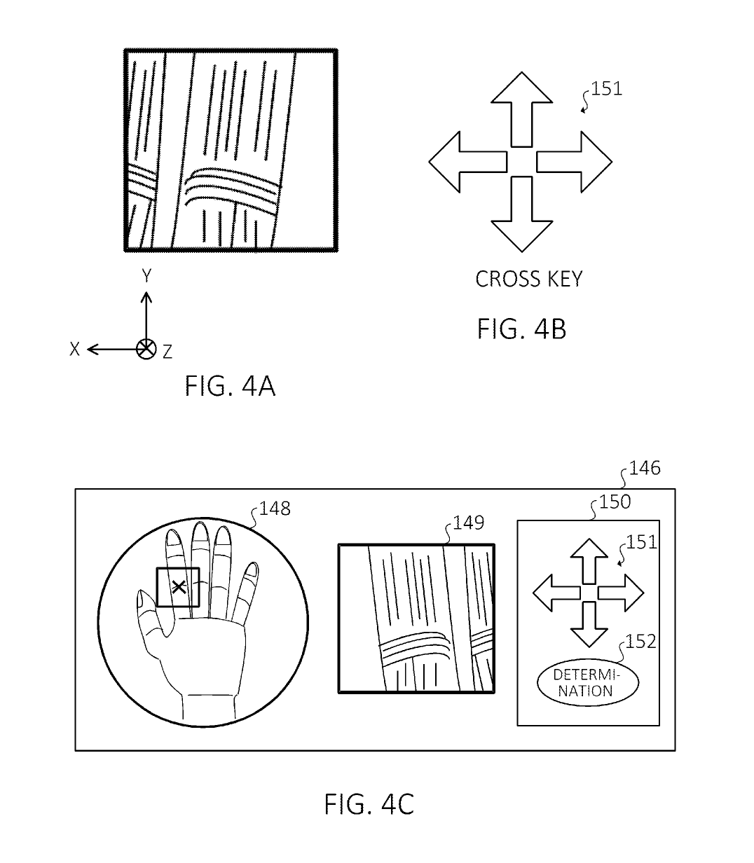

FIG. 4 shows an example of the designation of the measurement position for generating the photoacoustic image. The example in FIG. 4A is the example of the image in the case where the image is captured by the local imaging camera 122 at the position of the cursor 148b in FIG. 3A. The image in FIG. 4A corresponds to the XY plane, the lateral direction in FIG. 4 corresponds to the X-axis, and the vertical direction therein corresponds to the Y-axis. Note that the imaging direction of the local imaging camera 122 in FIG. 4A is the Z-axis positive direction, and the imaging direction is opposite to that of the image in FIG. 3A that is captured by the wide-view imaging camera 110 in the Z-axis negative direction. Accordingly, the image in FIG. 4A is inverted in the direction of the X-axis as compared with the image in FIG. 3A.

In Step S14, the designating unit 143 receives the designation of the measurement position that is performed by using a predetermined inputting unit from the user having referred to the displayed image of the local imaging camera 122. Herein, the designation of the measurement position may also be received via a cross key shown in FIG. 4B.

FIG. 4C shows an example of the display of the monitor 146. As shown in FIG. 4C, the image 148 by the wide-view imaging camera 110, an image 149 by the local imaging camera 122, and a position adjustment panel 150 are displayed. The position adjustment panel 150 includes a cross key 151 and a determination button 152. The position adjustment panel 150 is used for the input of the acquisition position of the local image when the image 148 by the wide-view imaging camera 110 is selected, and is used for the input of the measurement position when the image 149 by the local imaging camera 122 is selected. The cross key 151 is used for the change of the position of the local image or the measurement position. The determination button 152 is used for the determination of the local imaging position or the measurement position. Note that, in order to align the orientation of the image 149 with the orientation of the image 148, the image 149 is the image obtained by inverting the image in FIG. 4A in the X-axis direction by the image processing unit 142.

In the case where the user desires to measure the position away from the image displayed by the local imaging camera 122 in the Y-axis positive direction, the user presses an up button of the cross key 151. With this, the measurement position is moved in the Y-axis positive direction. In the case where the user desires to measure the position away from the image displayed by the local imaging camera 122 in the X-axis negative direction, the user presses a left button of the cross key 151. With this, the measurement position is moved in the X-axis negative direction. Thus, the user can finely adjust the measurement position with the cross key 151 while looking at the image displayed by the local imaging camera 122. Herein, the detailed designation of the measurement position is referred to as fine adjustment.

The designating unit 143 may gradually move the measurement position in accordance with a time period in which the button of the cross key 151 is pressed and held, or may move the measurement position by a predetermined distance every time the button of the cross key 151 is pressed. The user presses the determination button 152 when the measurement position has been moved to a de sired measurement position. With this, the measurement is started at the position corresponding to the imaging area of the local imaging camera 122, and the photoacoustic image is generated.

Note that the designation of the measurement position is not limited to the case where the measurement position is designated by using the position adjustment panel 150, and the measurement position may also be designated by using, e.g., a keyboard or a joystick. In addition, the measurement position may also be designated by dragging and dropping the image 149 in FIG. 4C. For example, in the case where the image 149 is dragged and dropped by a distance corresponding to half of the image 149 in the X-axis positive direction, the designating unit 143 may designate a position obtained by shifting the measurement position by the distance corresponding to half of the image 149 in the X-axis positive direction as the measurement position.

Note that, in Embodiment 2, a description will be given of an example in which the change of the measurement position is received in real time while the measurement is performed.

In Step S15, at the designated measurement position, each converting element 121 receives the photoacoustic wave generated from the inside of the hand by irradiation of the pulsed light, and generates the PA signal. The photoacoustic calculating unit 141 performs the generation of the photoacoustic image by using the PA signal acquired from each converting element 121.

Effect

Thus, the position designation of the imaging area of the local imaging camera 122 is performed based on the wide-view image of the object captured by the wide-view imaging camera 110 (coarse adjustment). Subsequently, the measurement position is designated based on the local image captured by the local imaging camera 122 (fine adjustment). With this, it becomes possible to easily designate the measurement position.

Note that, in the present embodiment, it has been described that, since the local imaging camera 122 is fixed to the supporter 120, the measurement position is designated while the supporter 120 is moved to implement the position designation in the local imaging camera 122, but the present invention is not limited thereto.

The area imaged by the local imaging camera 122 may be designated by using the actual coordinates (X, Y). For example, the imaging area may be changed by designating parameters of tilt and panning of the local imaging camera 122 that is fixed to a predetermined position and is capable of changing the imaging area by swiveling.

In addition, the imaging area may also be changed by providing a moving unit of the local imaging camera 122 separately from the moving mechanism 130 and causing the moving unit to move the local imaging camera 122 independently.

Further, the emission opening 133 does not need to move together with the supporter 120. For example, an emission opening moving mechanism separate from the support may be provided, or a mechanism that changes an emission direction by swiveling may be provided.

Designation of Measurement Area

Note that, as the measurement position in the above description, a relatively small region in which the acoustic wave adequate for generating a reconstructed image can be acquired without performing probe scanning, e.g., a mole with suspected melanoma in the hand of the object is assumed. However, in the present invention, it is also possible to designate the measurement area larger than the measurement position. For example, the image processing unit 142 displays a quadrangular frame indicative of the measurement area on the image 149 by the local imaging camera 122 in FIG. 4C. It is possible to change the size of the quadrangular frame by an operation by the user.

The designating unit 143 receives the designation of the measurement area in accordance with the quadrangular frame. Subsequently, the moving mechanism controlling unit 145 controls the moving mechanism 130 to move the supporter 120 to each position in the measurement area. Then, each of the converting elements 121 disposed all over the inside of the cup-shaped supporter 120 receives the acoustic wave at each position. Subsequently, the photoacoustic calculating unit 141 generates the photoacoustic image based on the acoustic wave received by each converting element 121.

It is also possible to perform enlargement/reduction of the measurement area. For example, the designating unit 143 may receive the enlargement and reduction of the measurement area by movement by dragging and dropping of each side of the quadrangular frame. In the case where the size of the quadrangular frame displayed on the image 149 in FIG. 4C is changed, the designating unit 143 may change the size of the measurement area in accordance with the changed quadrangular frame.

In addition, the acoustic wave measuring apparatus 100 may also perform the measurement in the measurement area by repeatedly performing the irradiation of the pulsed light and the reception of the acoustic wave at regular intervals while causing the supporter 120 to scan in the measurement area at a predetermined speed. For example, in the case where the designated measurement area is rectangular, the moving mechanism controlling unit 145 causes the supporter 120 to scan in the rectangle such that the rectangle is irradiated with the pulsed light. The light source 131 irradiates the object (hand) with the pulsed light at regular intervals also during a time period in which the supporter 120 moves in the rectangle. The photoacoustic calculating unit 141 generates the photoacoustic image based on the acoustic wave received by each converting element 121 at each position to which the converting element 121 has moved.

Further, the designating unit 143 may receive the designation of the measurement area that is larger than the area of the image captured by the local imaging camera 122. For example, in the image 149 in FIG. 4C, the designating unit 143 receives the designation of four points in the image 149 from the user while the imaging area of the image 149 is moved by the operation of the cross key 151. For example, the designating unit 143 may receive the designation of each point via the determination button 152. Subsequently, the designating unit 143 may receive a quadrangle obtained by connecting the received four points as the measurement area. Note that the designation of the measurement area is not limited to the case where the measurement area is designated by using the quadrangle, and a polygon may also be received as the measurement area.

In addition, the designating unit 143 may receive the designation of the measurement area in a screen on which the photoacoustic image is displayed. Specifically, the designating unit 143 may receive the designation in which the next measurement area is shifted from the current measurement area in the X-axis direction and the Y-axis direction based on the screen on which the photoacoustic image is displayed. For example, the designating unit 143 may display the cross key 151 shown in FIG. 4B at the side of the screen of the photoacoustic image, and receive the designation of the next measurement area.

Thus, by the acoustic wave measuring apparatus 100 shown in Embodiment 1, there is obtained an effect that it is possible to easily designate the measurement area.

### Embodiment 2

### Real-Time Position Designation

A description will be given of the case where the wide-view imaging camera 110 and the local imaging camera 122 perform the imaging in real time also during the photoacoustic measurement. The acoustic wave measuring apparatus 100 is capable of changing the measurement position based on the screen of the wide-view imaging camera 110 or the local imaging camera 122 while displaying the photoacoustic image.

For example, as shown in FIG. 3B, when the position of the cursor 148b is moved, the moving mechanism controlling unit 145 moves the supporter 120 to the position corresponding to the moved cursor 148b. Also during a time period in which the supporter 120 is moved, the object (hand) is irradiated with the pulsed light at regular intervals, and the photoacoustic calculating unit 141 performs the generation of the photoacoustic image data based on the PA signal received by the converting element 121 at regular intervals. The image processing unit 142 updates the photoacoustic measurement screen based on the generated photoacoustic image data whenever necessary. With this, in the image processing unit 142, the photoacoustic image is displayed in real time also during the movement of the supporter 120.

In addition, it is also possible to designate the measurement position based on the photoacoustic measurement screen in real time. For example, the image processing unit 142 displays a predetermined blood vessel as the photoacoustic image. In the case where the user desires to measure the position of the blood vessel that is moved in the X-axis positive direction, the designating unit 143 receives the designation of the measurement position that is moved from the current measurement position in the X-axis positive direction to thereby designate the position of the blood vessel in the X-axis positive direction as the next measurement position. During a time period in which the moving mechanism 130 moves the supporter 120 in the X-axis positive direction, the object (hand) is irradiated with the pulsed light at regular intervals, and the photoacoustic calculating unit 141 performs the generation of the photoacoustic image data based on the PA signal received by the converting element 121 at regular intervals in real time. With this, the image processing unit 142 displays the image of the blood vessel at the position in the X-axis positive direction as the photoacoustic image.

Thus, according to the present embodiment, also during the time period in which the supporter 120 is moved to the measurement position, it is possible to perform the measurement in real time. As a result, the user can designate or correct the measurement position appropriately while checking the photoacoustic image.

In addition, the photoacoustic calculating unit 141 may generate the photoacoustic image data per pulsed light, and cause the image processing unit 142 to display the photoacoustic image data. The acoustic wave measuring apparatus 100 may receive the position where the photoacoustic image data per pulsed light is generated while displaying the image in the local imaging camera 122.

In the above embodiment, an example in which the object is the palm of the hand has been described, but the present invention is not limited thereto. For example, the object may be the bottom of the foot. In addition, the object may also be the breast. Further, a small animal such as a mouse or a rat may be used as the object.

In the above embodiment, the imaging direction by the wide-view imaging camera 110 is opposite to the imaging direction by the local imaging camera 122, but the present invention is not limited thereto. For example, the wide-view imaging camera 110 may image the object from an oblique direction.

In addition, the object may be imaged from different angles by the wide-view imaging camera 110 and the local imaging camera 122 by installing the wide-view imaging camera 110 on the same side (e.g., lower side) as the local imaging camera 122 relative to the hand serving as the object. For example, a plurality of the wide-view imaging cameras 110 are disposed at predetermined positions behind the supporter 120, and an image generating unit that is not shown generates a combined image using the plurality of the wide-view imaging cameras 110. The combined image corresponds to the wide-view image (an image of a first area) in the present embodiment. At this point, it is preferable to perform image processing that does not display the supporter 120 in the combined image. Similarly to the image processing unit 142, the image generating unit can be implemented by the information processing apparatus with image processing capability.

Note that the description has been given of the example in which the plurality of the wide-view imaging cameras 110 are used, but the present invention is not limited thereto. For example, the acoustic wave measuring apparatus 100 causes one wide-view imaging camera 110 to image the object from the oblique direction such that the object is not hidden by the supporter 120. Subsequently, the image processing unit 142 may generate the wide-view image by correcting the distortion of the image captured by the wide-view imaging camera 110 from the oblique direction.

Note that the acoustic wave measuring apparatus 100 may change only the position that is visualized without moving the supporter 120.

The present invention can be implemented by a system or a computer of an apparatus (or a device such as a CPU or an MPU) that implements the functions of the above-described embodiments by reading and executing a program recorded in a storage apparatus. In addition, the present invention can also be implemented by a method including steps executed by the system or the computer of the apparatus that implements the functions of the above-described embodiments by reading and executing the program recorded in the storage apparatus. Further, the present invention can be implemented by a circuit (e.g., an ASIC) that implements one or more functions. In order to achieve this object, the above program is provided to the above computer, e.g., through networks or from various types of recording media (i.e., computer-readable recording media in which data is stored non-transitorily) that can be the above storage apparatuses. Consequently, the above computer (including the device such as the CPU or the MPU), the above method, and the above program (including a program code and a program product) are included in the scope of the present invention. A computer-readable recording medium in which the above program is stored non-transitorily is also included in the scope of the present invention.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2017-012671, filed on Jan. 27, 2017, which is hereby incorporated by reference herein in its entirety.

What is claimed is:

1. An acoustic wave measuring apparatus for generating a characteristics information distribution of an inside of an object by using an acoustic wave propagating from the object,
the acoustic wave measuring apparatus comprising:
a receiving unit configured to receive the acoustic wave;
a first imaging unit configured to image a first area of the object from a first direction, and acquire an image of the first area;
a second imaging unit configured to image an area smaller than the first area of the object from a second direction different from the first direction, and acquire a local image;
a designating unit configured to receive, from a user, designation of a position where the imaging is performed by the second imaging unit in the image of the first area captured by the first imaging unit, and receive, from the user, designation of a second area, the characteristics information distribution of which is generated in the local image captured by the second imaging unit; and
a generating unit configured to generate the characteristics information distribution by using the acoustic wave received by the receiving unit.

2. The acoustic wave measuring apparatus according to claim 1, wherein
the second imaging unit is installed on aside opposite to the first imaging unit when viewed from the object, and the second imaging unit is configured to image the object from the second direction opposite to the first direction.

3. The acoustic wave measuring apparatus according to claim 1, wherein the first imaging unit is configured to include an imaging unit that is installed on the same side as the second imaging unit when viewed from the object,
the acoustic wave measuring apparatus further comprising:
an image generating unit configured to generate the image of the first area, based on an image acquired by the imaging unit included in the first imaging unit.

4. The acoustic wave measuring apparatus according to claim 1, further comprising:
a position moving unit configured to move the receiving unit to a position corresponding to the area, the designation of which is received from the user and the characteristics information distribution of which is to be generated.

5. The acoustic wave measuring apparatus according to claim 4, further comprising:
a supporting unit configured to support the receiving unit and the second imaging unit, wherein
the position moving unit is configured to change a position of the supporting unit relative to the object, and
the receiving unit is configured to receive the acoustic wave generated in the area imaged by the second imaging unit.

6. The acoustic wave measuring apparatus according to claim 1, further comprising:
a light irradiating unit configured to irradiate the object with light, wherein
the acoustic wave is a photoacoustic wave propagating from the object irradiated with the light.

7. The acoustic wave measuring apparatus according to claim 5, further comprising:
a light irradiating unit configured to be supported by the supporting unit, and irradiate the object with light, wherein
the acoustic wave is a photoacoustic wave propagating from the object irradiated with the light.

8. The acoustic wave measuring apparatus according to claim 1, further comprising:
an inputting unit configured to receive at least one of an input of the position where the imaging is performed by the second imaging unit in the image of the first area and an input of the area, the characteristics information distribution of which is generated in the local image.

9. The acoustic wave measuring apparatus according to claim 4, wherein
the generating unit is configured to generate the characteristics information distribution also during a time period in which the receiving unit is moved by the position moving unit.

10. A control method for an acoustic wave measuring apparatus for generating a characteristics information distribution of an inside of an object by using an acoustic wave propagating from the object, the acoustic wave measuring apparatus including:
a receiving unit that receives the acoustic wave;
a first imaging unit that images a first area of the object from a first direction, and acquires an image of the first area; and
a second imaging unit that images an area smaller than the first area of the object from a second direction different from the first direction, and acquires a local image,
the control method comprising:
receiving, from a user, designation of a position where the imaging is performed by the second imaging unit in the image of the first area captured by the first imaging unit, and receiving, from the user, designation of a second area, the characteristics information distribution of which is generated in the local image captured by the second imaging unit; and
generating the characteristics information distribution by using the acoustic wave received by the receiving unit.
